(19) United States
(12) Patent Application Publication
(10) Pub. No.: US 2008/0172054 A1
(43) Pub. Date: Jul. 17, 2008

(54) ORTHOPEDIC DEVICE FOR SECURING TO TISSUE

(75) Inventors: Jody L. Claypool, Columbia City, IN (US); Brian H. Thomas, Columbia City, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER TECHNOLOGY, INC., Warsaw, IN (US)

(21) Appl. No.: 11/623,489

(22) Filed: Jan. 16, 2007

(51) Int. Cl. A61B 17/15

(52) U.S. Cl. 606/87; 606/96

(57) ABSTRACT

An orthopedic device including a base and an adhesive pad attached to the base. The adhesive pad receives an adhesive capable of securing the orthopedic device to tissue. The orthopedic device may also include an attachment mechanism for receiving and attaching additional orthopedic components and/or surgical instruments thereto. For example, a cut guide, a pin placer, or another orthopedic component or surgical instrument may be secured to the attachment mechanism of the orthopedic device of the present invention.

### BACKGROUND

1. Field of the Invention

The present invention relates to an orthopedic device, and, more particularly, to an orthopedic device configured to be secured to tissue.

2. Description of the Related Art

Various orthopedic devices are designed to be fixed to tissue within a patient's body. These orthopedic devices may be fixed to tissue by cerclage wire, bone screws, or pins. Once fixed, the orthopedic devices may perform a set function, such as guiding a surgical saw to resect bone, and/or may provide a stable base for the attachment and alignment of additional orthopedic components. For example, once an orthopedic device is fixed to tissue, such as bone, an additional orthopedic component, such as a cut guide, may be attached to the orthopedic device and aligned relative thereto.

### SUMMARY

The present invention relates to an orthopedic device, and, more particularly, to an orthopedic device configured to be secured to tissue. In one exemplary embodiment, the orthopedic device includes a base and an adhesive pad attached to the base. The adhesive pad receives an adhesive capable of securing the orthopedic device to tissue. The orthopedic device may also include an attachment mechanism for receiving and attaching additional orthopedic components and/or surgical instruments thereto. For example, a cut guide, a pin placer, or another orthopedic component or surgical instrument may be secured to the attachment mechanism of the orthopedic device of the present invention. The orthopedic device may also include a power source and a switch. Upon movement of the switch, the power source may be connected to curing means, such as an ultraviolet light, to speed curing of the adhesive. In another exemplary embodiment, the orthopedic device contains a rupturable pouch. The pouch may contain an adhesive, or a catalyst such as a compound to speed curing of a previously applied adhesive, which is released when the pouch is ruptured.

Advantageously, the design of the present orthopedic device eliminates the need to use screws, pins, or wires to secure the orthopedic device to tissue. As a result, surgical time is reduced, fewer surgical instruments are utilized, and the complexity of the surgical procedure is lessened. Additionally, when curing means are used, the surgeon can immediately initiate rapid curing of the adhesive to further reduce surgical time. The orthopedic device may be designed to be disposable, eliminating the cost and complexity of additional sterilization procedures and lowering manufacturing costs. Moreover, the present orthopedic device provides the surgeon with the ability to secure the orthopedic device to tissue with infinite control in multiple degrees of freedom.

In one form thereof, the present invention provides an orthopedic system including, an orthopedic device including a base, an adhesive pad attached to the base, a quantity of medical grade adhesive associated with the adhesive pad, the quantity of adhesive being sufficiently strong to secure the base to a portion of tissue by itself.

In another form thereof, the present invention provides an orthopedic system including an orthopedic device including a base, an adhesive pad attached to the base, a quantity of medical grade adhesive associated with the adhesive pad, the quantity of adhesive being sufficiently strong to secure the base to a tissue by itself; and one of a surgical instrument and an orthopedic component configured to be secured to the orthopedic device.

In another form thereof, the present invention provides a method for securing an orthopedic system to tissue, including the steps of applying an adhesive to an adhesive pad attached to a base, positioning the base on the tissue, curing the adhesive to secure the adhesive pad to the tissue, and attaching one of a surgical instrument and an orthopedic component to the base.

### BRIEF DESCRIPTION OF THE DRAWINGS

The above-mentioned and other features and advantages of this invention, and the manner of attaining them, will become more apparent and the invention itself will be better understood by reference to the following descriptions of embodiments of the invention taken in conjunction with the accompanying drawings, wherein:

FIG. 1 is a perspective view of the orthopedic device of the present invention with a cut guide attached thereto;

FIG. 2 is an exploded perspective view of the orthopedic device and cut guide of FIG. 1;

FIG. 3 is a cross-sectional view of the orthopedic device of FIG. 1, taken along line 3-3 of FIG. 1; and

FIG. 4 is a cross-sectional view similar to FIG. 3 of an orthopedic device according to another embodiment.

Corresponding reference characters indicate corresponding parts throughout the several views. The exemplifications set out herein illustrate exemplary embodiments of the invention and such exemplifications are not to be construed as limiting the scope of the invention any manner.

### DETAILED DESCRIPTION

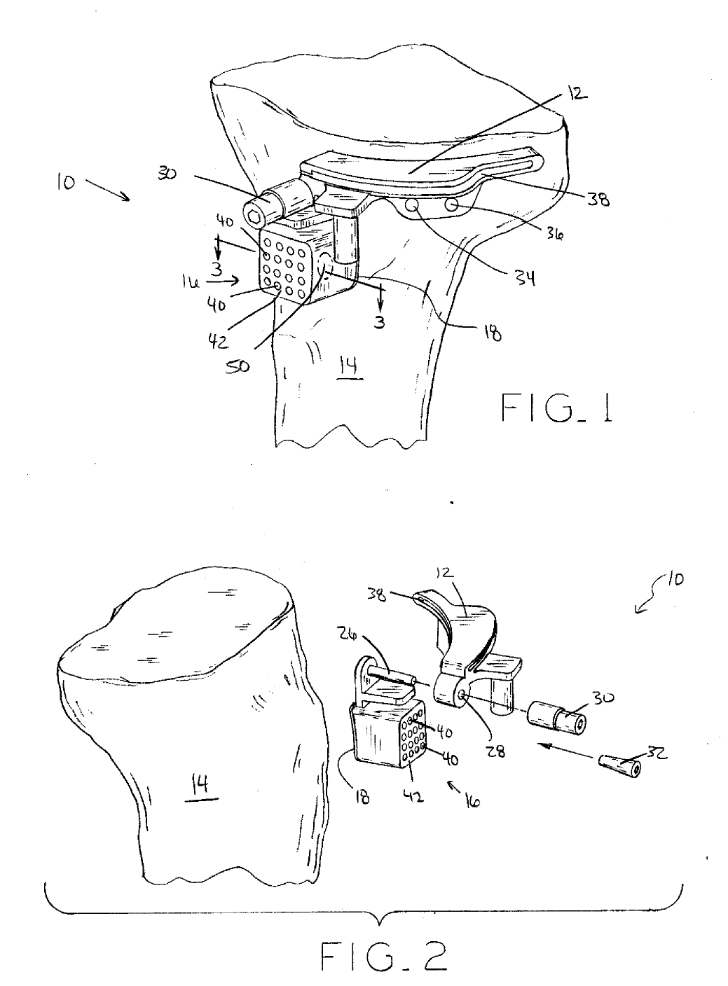

As shown in FIGS. 1 and 2, orthopedic device 10, which includes base 16 and adhesive pad 18, is secured to tibia 14. While orthopedic device 10 is described and depicted herein as secured to tibia 14, orthopedic device 10 may be secured in other locations and to other types of tissue in accordance with the teachings herein. For example, orthopedic device 10 may be secured to other bones including the pelvis, femur, humerus, ulna, radius, tarsus, metatarsus, scapula, clavicle, fibula, talus, vertebral bodies, and phalanges. Base 16 of orthopedic device 10 may be designed to match the shape or contour of tissue generally, such as tibia 14, or a specific portion of tissue, such as the intercondylar notch of the femur. In one exemplary embodiment, adhesive pad 18 is a distinct component and is attached to base 16 in a conventional manner, such as by gluing. In another exemplary embodiment, adhesive pad 18 is attached to base 16 by forming orthopedic device 10 as a monolithic structure. In this embodiment, adhesive pad 18 defines the tissue engaging portion of orthopedic device 10 and is not a separate component thereof.

Adhesive pad 18 receives adhesive 20 (FIG. 4) for securing orthopedic device 10 to tissue. Adhesive 20 may be any known medical grade adhesive having sufficient strength to secure adhesive pad 18 and base 16 directly to tissue. For example, depending on the material used to form orthopedic device 10 and the type of tissue selected, light curable acrylic adhesives, acrylic adhesives, cyanoacrylate adhesives, silicone adhesives, urethane adhesives, and/or epoxy adhesives may be utilized. Some of these adhesive types are commercially available, such as Transbond XT, manufactured by 3M Unitek; Loctite 3552, 3553, 3011, 3051, 3091, 3201, 3211, 3301, 3311, 3321, 3341, 3381, 3345, 4304, and 4305, manufactured by Henkel KGaA; Python Light Cure Adhesive and Turbon Bond Light Cure Bonding Adhesive manufactured by TP Orthodontics; Illuminate Light Cure Adhesive manufactured by Otho Organizers; Ultra Band-Lok manufactured by GAC International; FlowTrain manufactured by Reliance Orthodontic Products; Cure and Pro Lock manufactured by All-Star Orthopedics; Eagle No Drift Adhesive manufactured by American Orthodontics; ConTec LC manufactured by Dentaurum, Inc.; Econo No-Mix manufactured by Dentsply; Champion Light Cure Orthodontic Adhesive Kit manufactured by Sullivan-Schein Dental; Granitec M5 and Light Cured Orthodontic Adhesive manufactured by Orthodontic Supply and Equipment Co.; Adhere LC manufactured by Ortho Specialties; Mini-Mold Paste and Ultra Brand-lok manufactured by Ortho Arch Co.; Enlight Bonding System manufactured by Ormco Corp.; and Easy Bond Light Cure manufactured by Nexadental. Additionally, adhesive 20 may be in sheet form, such as an adhesive tape, and/or in liquid form.

In one exemplary embodiment, shown in FIG. 4, adhesive pad 18 is non-porous. In this embodiment, adhesive 20 is received on adhesive pad 18. For example, adhesive pad 18 may receive adhesive 20 thereon prior to positioning orthopedic device 10 adjacent tissue. Additionally, adhesive 20 may be placed directly on tibia 14 and, with adhesive pad 18 of orthopedic device 10 positioned adjacent tibia 14, adhesive pad 18 receives adhesive 20 thereon.

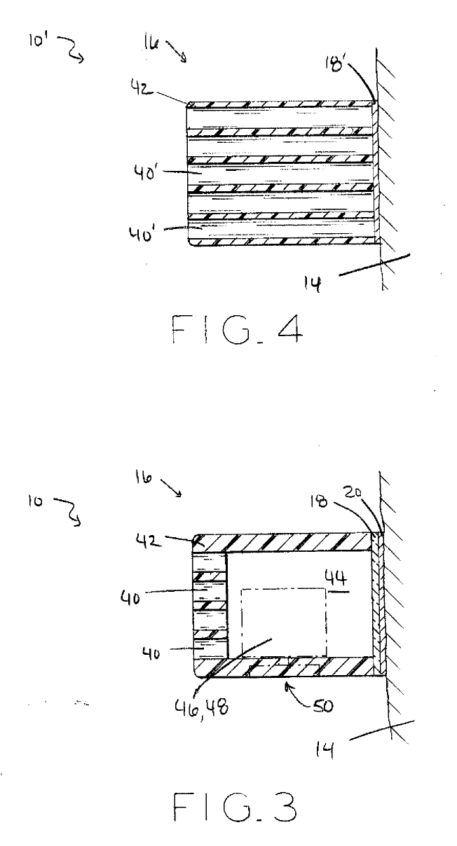

In another exemplary embodiment, the adhesive pad is porous. This embodiment, depicted as orthopedic device 10′ in FIG. 3, has several features which are identical to orthopedic device 10 of FIGS. 1, 2, and 4, discussed in detail herein, and identical reference numerals have been used to identify identical or substantially identical features therebetween. As shown in FIG. 3, adhesive 20 (not shown) is received within adhesive pad 18′, allowing adhesive pad 18′ to directly contact tibia 14. For example, adhesive 20 may be received within adhesive pad 18′ prior to packaging orthopedic device 10′. A protective cover may then be placed over adhesive pad 18′ to protect the same until orthopedic device 10′ is ready for use. To utilize orthopedic device 10′, a surgeon removes orthopedic device 10′ from its packaging and then removes the protective covering from adhesive pad 18′. Orthopedic device 10′ is then ready to be secured to tibia 14. In another exemplary embodiment, adhesive 20 may be placed directly on tibia 14 and, when adhesive pad 18′ of orthopedic device 10′ is positioned adjacent tibia 14, adhesive pad 18′ absorbs adhesive 20 to receive adhesive 20 therein.

With orthopedic device 10 properly positioned adjacent tibia 14, and adhesive 20 received by adhesive pad 18 as described in detail above, curing means may be used to facilitate curing of adhesive 20 and to rigidly fix orthopedic device 10 to tibia 14. For example, adhesive 20 may be time curing, i.e., the passage of a predetermined period of time results in the curing of adhesive 20. Adhesive 20 may also be designed such that the use of additional curing means facilitates the curing of adhesive 20. For example, exposure to air, ultraviolet light, visible light, thermal energy, microwaves, electrical current, or chemical initiators may result in curing of adhesive 20. In one exemplary embodiment, adhesive 20 has sufficient strength to retain orthopedic device 10 against tissue, but does not immediately begin to cure. In this embodiment, the surgeon may position orthopedic device 10 on tibia 14 for preliminary testing and, when the desired position is located, apply curing means to facilitate the curing of adhesive 20. Once adhesive 20 has cured, orthopedic device 10 is firmly secured in the desired position.

As shown in FIGS. 1, 2, and 4, base 16 includes apertures 40 which may facilitate curing of adhesive 20. For example, when the curing of adhesive 20 is facilitated by exposure to ultraviolet light, apertures 40 will allow ultraviolet light to travel through end 42 of base 16 into cavity 44 (FIG. 4). The light may then travel through cavity 44 toward adhesive pad 18 and adhesive 20. In this embodiment, an external source of ultraviolet light may be placed near end 42 of base 16 to facilitate the curing of adhesive 20. Adhesive pad 18 may be translucent to allow ultraviolet light to pass therethrough and to interact with adhesive 20. In another exemplary embodiment, both base 16 and adhesive pad 18 are translucent to allow the passage of light therethrough and eliminate the need for apertures 40. In another exemplary embodiment, depicted in FIG. 3, apertures 40′ extend from end 42 of base 16 all the way to adhesive pad 18′. In this embodiment, apertures 40′ channel ultraviolet light, for example, directly to adhesive pad 18′. In another exemplary embodiment, a light pipe, such as a fiber optic strand or rope, may be used to channel light, internally or externally of base 16, directly to adhesive 20. A light pipe may also be utilized in conjunction with apertures 40, 40′.

As shown in FIG. 4, base 16 may include cavity 44 formed therein. In one exemplary embodiment, power source 46, e.g., a battery, and curing means 48, e.g., an LED light, are positioned within cavity 44 of base 16. Curing means 48 may be any device capable of facilitating the curing of an adhesive. For example curing means 48 may generate ultraviolet light, visible light, thermal energy, microwaves, or electrical current, or may release chemical initiators. Power source 46 is connectable to curing means 48 by movement of a switch, such as button 50. When button 50 is depressed, power source 46 is connected to curing means 48 to facilitate curing of adhesive 20. Thus, if curing of adhesive 20 is facilitated by exposure to ultraviolet light, an ultraviolet light source may be connectable to power source 46 when button 50 is depressed. This allows orthopedic device 10 to have a self-contained curing means and eliminates the need for any additional equipment or instruments to properly secure orthopedic device 10 to tibia 14.

In another exemplary embodiment, a rupturable pouch (not shown) may be positioned within cavity 44. In one exemplary embodiment, the rupturable pouch contains adhesive 20. Button 50 may include a pin or other rupture apparatus which, when button 50 is depressed, ruptures the pouch. Adhesive 20 is then be received by porous adhesive pad 18, shown in FIG. 3, or passes through apertures (not shown) in adhesive pad 18′ and is retained between adhesive pad 18′ and tibia 14. In another exemplary embodiment, the rupturable pouch contains curing means, such as a chemical initiator therein. Similar to the exemplary embodiment discussed above, depressing button 50 ruptures the pouch, allowing curing means to contact adhesive 20 received by adhesive pad 18′ or to pass through apertures in adhesive pad 18 to contact adhesive 20 between adhesive pad 18 and tibia 14.

As depicted in FIG. 2, orthopedic device 10 includes attachment means, depicted as pin 26, for attachment to a surgical instrument and/or an orthopedic component. For example, pin 26 may be received within aperture 28 of tibial cut guide 12, which is configured to be secured to orthopedic device 10. Once attached to orthopedic device 10, tibial cut guide 20 may then be utilized to guide a cut during the orthopedic surgery. In addition, the attachment means may be configured for attachment to other orthopedic components, such as different cut guides or pin placers. While the attachment means is depicted as pin 26, any known mechanism which allows for connection of orthopedic device 10 to another orthopedic component and/or instrument may be used. In this embodiment, knob 30 may be received on pin 26 to adjust the varus/valgus position of cut guide 12 and, ultimately, retain cut guide 12 in the desired varus/valgus position. Once the varus/valgus position of cut guide 12 is set, conical screw 32 may be screwed into base 16 to increase the posterior slope of cut guide 12. Once cut guide 12 is properly aligned relative to orthopedic device 10, stabilizing pins may be inserted through apertures 34, 36 (FIG. 1) of cut guide 12. Slot 38 of cut guide 12 is sized to receive an orthopedic saw therethrough to resect the proximal portion of tibia 14. In another exemplary embodiment, a surgical instrument or orthopedic component is integral with orthopedic device 10, eliminating the need for attachment means.

While this invention has been described as having preferred designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An orthopedic system comprising:
an orthopedic device including:
a base;
an adhesive pad attached to said base,
a quantity of medical grade adhesive associated with said adhesive pad, said quantity of adhesive being sufficiently strong to secure said base to a portion of tissue by itself.

2. The orthopedic system of claim 1, wherein said tissue is a bone selected from the group consisting of the tibia, pelvis, femur, humerus, ulna, radius, tarsus, metatarsus, scapula, clavicle, fibula, talus, vertebral bodies, and phalanges.

3. The orthopedic system of claim 1, wherein said adhesive is received by said adhesive pad prior to securing said base to said tissue.

4. The orthopedic system of claim 1, further comprising a cut guide, said cut guide configured to be selectively secured to said orthopedic device.

5. The orthopedic system of claim 1, wherein said base further includes a power source and a switch, said power source connectable to curing means to cure said adhesive upon movement of said switch.

6. The orthopedic system of claim 5, wherein said curing means is an ultraviolet light source.

7. The orthopedic system of claim 1, wherein said adhesive is selected from the group consisting of acrylics, cyanoacrylates, silicones, urethanes, and epoxies.

8. The orthopedic system of claim 1, wherein said base further includes apertures extending through said base, said apertures configured to allow light to reach said adhesive pad, whereby light received within said apertures facilitates curing of said adhesive.

9. The orthopedic system of claim 8, wherein said adhesive pad is translucent.

10. The orthopedic system of claim 1, wherein said adhesive pad is integral with said base.

11. An orthopedic system comprising:
an orthopedic device including:
a base;
an adhesive pad attached to said base,
a quantity of medical grade adhesive associated with said adhesive pad, said quantity of adhesive being sufficiently strong to secure said base to a tissue by itself; and
one of a surgical instrument and an orthopedic component configured to be secured to said orthopedic device.

12. The orthopedic system of claim 11, wherein said tissue is a bone selected from the group consisting of the tibia, pelvis, femur, humerus, ulna, radius, tarsus, metatarsus, scapula, clavicle, fibula, talus, vertebral bodies, and phalanges.

13. The orthopedic system of claim 11, wherein said orthopedic device further comprises curing means for facilitating the curing of said quantity of adhesive.

14. The orthopedic system of claim 11, wherein said one of said surgical instrument and an orthopedic component comprises a cut guide.

15. The orthopedic system of claim 11, wherein said one of said orthopedic instrument and said orthopedic tool is integral with said orthopedic device.

16. A method for securing an orthopedic system to tissue, comprising the steps of:
applying an adhesive to an adhesive pad attached to a base;
positioning the base on the tissue;
curing the adhesive to secure the adhesive pad to the tissue; and
attaching one of a surgical instrument and an orthopedic component to the base.

17. The method of claim 16, further comprising the step of utilizing the orthopedic component during orthopedic surgery.

18. The method of claim 16, wherein the tissue is a bone selected from the group consisting of the tibia, pelvis, femur, humerus, ulna, radius, tarsus, metatarsus, scapula, clavicle, fibula, talus, vertebral bodies, and phalanges.

19. The method of claim 16, wherein the orthopedic component is selected from the group consisting of a cut guide and a pin placer.

20. The method of claim 16, further comprising, after the positioning step, the step of adjusting the position of the orthopedic component relative to the base and the tissue.
